(19) United States
(12) Patent Application Publication
(10) Pub. No.: US 2021/0177564 A1
(43) Pub. Date: Jun. 17, 2021

(54) PERMANENT, REMOVABLE AND SELF-SUSTAINED URETHRAL PROBE

(71) Applicants: Salvador Vilar Correia Lima, Recife (BR); Fabio De Oliveira Vilar, Recife (BR)

(72) Inventor: Salvador Vilar Correia Lima, Recife (BR)

(73) Assignees: Salvador Vilar Correia Lima, Recife, PE (BR); Fabio De Oliveira Vilar, Recife, PE (BR)

(21) Appl. No.: 16/711,010

(22) Filed: Dec. 11, 2019

(51) Int. Cl. A61F 2/00; A61F 2/04

(52) U.S. Cl. CPC A61F 2/0009; A61F 2002/047; A61F 2/04

(57) ABSTRACT

A permanent, removable and self-sustainable urethral probe, is provided. The probe includes a tubular body, which includes two discs, one being a proximal fixed disc and the other being a distal mobile disc. The tubular body has a distal end that includes holes connected by a longitudinal channel traversing the tubular body. The probe includes a connector that may be closed by a corresponding cover. The total length of this probe 1 is 7 cm for child models and 8 cm for adult models. A 15 cm is also available to be used in patients with special needs such as wheelchairs.

### BACKGROUND

### 1. Field of the Invention

This invention patent application relates to a particular type of urethral probe, which is now part of the field of hospital/medical use devices and items. This permanent, removable and self-sustainable urethral probe is to be used on living human male and female patients suffering from urethral dysfunction, incontinence and chronic urinary retention, among other secondary causes that affect bladder function in order to provide a natural method for bladder emptying, emulating voiding physiology.

### 2. Description of Related Art

Permanent or intermittent bladder catheterization, along with the use of diapers, have been featured as viable alternatives for patients with bladder emptying dysfunctions, either caused by permanent loss of urination or poor, or lack of, bladder emptying.

Reduction or lack of urethral resistance at any stage of life, in particular on children suffering from neurological diseases, causes the bladder to lose the capacity of storing urine and, therefore, become dysfunctional, leading to atrophy of detrusor muscles and reduction, loss or increase in stimulation from the autonomic nervous system.

Patients suffering reduction or loss of urethral resistance require reinforcing mechanisms in order to stimulate the bladder's neuromuscular system and enable proper urine storage, since the patents that are unable to empty their bladders require mechanisms to make this process viable.

Intermittent catheterization is the most commonly used method for this purpose, with its main inconvenience in that it must be carried out several times during the day, as well as the amount of material required for such (Mazouni et al., 2004).

Urinary incontinence and retention in patients from all age brackets pose a challenging issue for the urological community, particularly in cases where the use of surgically implantable devices cannot be applied, either due to the presence of co-morbidities such as diabetes, severe neurological lesion, low age or elderly patients suffering from said anomalies.

The difficulty of self-catheterization for children and elderly people, particularly female patients, leads to improper bladder emptying which, in turn, leads to damages to the urinary tract and a subsequent renal failure condition, eventually leading to irreversible situations, which may require transplantation or hemodialysis (Ricciotti et al., 1995).

The simplest form of permanent bladder emptying is the Foley probe, which includes an inflatable balloon connected to a collector bag, which is periodically emptied.

Despite properly emptying the bladder, other features of the inflatable balloon such as size, commonly used manufacturing material, as well as the inconvenience itself of an installed inflatable balloon, render it unable to be used in a permanent condition in the aforementioned situations.

Some intra-urethral devices were previously described but failed to prove useful in solving the issue (Yachia, 1997).

In general, it has been determined by the present disclosure that probes belonging to the state of the art are not perfectly adjustable and are not designed for optimal fitting to the genitourinary anatomy.

### SUMMARY

In face of the aforementioned drawbacks, the permanent, removable and self-sustainable urethral probe was developed, in order to address compactness requisites, featuring a reduced size compared to similar devices belonging to the state of the art; it is also adjustable and designed for perfect fitting to the genitourinary anatomy.

In face of the particularities of the aforementioned state of the art, the probe herein proposed was developed, manufactured in medical grade silicone and including two discs with a central protuberance, one being a proximal fixed disc coupled to the bladder neck and providing occlusion, and the other being a distal sliding disc which, upon adjustment to the external female meatus, or male perineal urethrostomy, provides full urethral occlusion and subsequent continence.

The probe at hand is defined as a tubular body featuring four holes on its proximal end which has 2.5 cm in length

The tubular body is manufactured in flexible and transparent medical grade silicone with a smooth surface and having rounded end (proximal end) and featuring a small 1 mm diameter hole to allow passage of a guide wire, which may prove necessary in some situations to provide easier introduction.

The distal end includes a connector with a cover and an open-close mechanism, which enables bladder emptying whenever required. (FIGS. 1, 2 and 3).

The probe includes a disposable chuck made of polypropylene (FIGS. 4A and 4B), which shall enable introduction of the probe through the patient's bladder. It comes in 2 sizes to enable safe introduction of the probe on its different sizes.

This chuck includes a channel with a 1 mm diameter opening through its entire length, in order to enable potential use of the guide wire, as previously mentioned.

The innovations of this invention are its reduced and adjustable size, and its perfect fitting to the genitourinary anatomy.

There is a special sized probe with an extension of 15 cm between disks to be used in patients who use wheelchairs and are unable to reach and adequately manipulate the device. This chuck is part of the pack that comes with the specific probe and is also made of polypropylene.

The exit ports of the existing probe in the prior art are insufficient in specific cases where the elimination of crystals produced by the patient's kidneys is required.

Another drawback noted is the difficulty of positioning the tube through the patient's bladder and, in addition, the lack of ergonomics of the tube in cases where the patients are wheelchair-bound, as these individuals usually have disability properly reach and handle the device.

The purpose of this patent application is to meet the aforementioned needs and provide patients with a urethral probe that provides greater comfort by having a compact and adjustable dimension perfectly to the genitourinary anatomy as well as providing a disposable mandrel or chuck, which greatly assists in positioning the said rig and finally, to serve the wheelchair patients appropriately, offering a model with dimensions that facilitate the correct manipulation by the wheelchair.

### BRIEF DESCRIPTION OF THE DRAWINGS

This invention patent application will be described in detail with reference to the drawings listed below, in which:

FIG. 1 shows a perspective view of a permanent, removable and self-sustainable urethral probe according to the present disclosure;

FIG. 2 shows a side view of the probe of FIG. 1.

FIG. 3 shows the probe of FIG. 1 as inserted through the urethra of a patient and reaching the bladder.

FIGS. 4A and 4B show, respectively, the probe described herein, including the chuck, and the aforementioned probe with said chuck alongside it, in order to enable introduction of the probe through the patient's urethra.

FIG. 5 shows the use of the permanent, removable and self-sustained urethral probe.

FIG. 6 shows a side view of the wheelchair developed probe where readings/distances of progression to the distal sliding disc are indicated.

### DETAILED DESCRIPTION

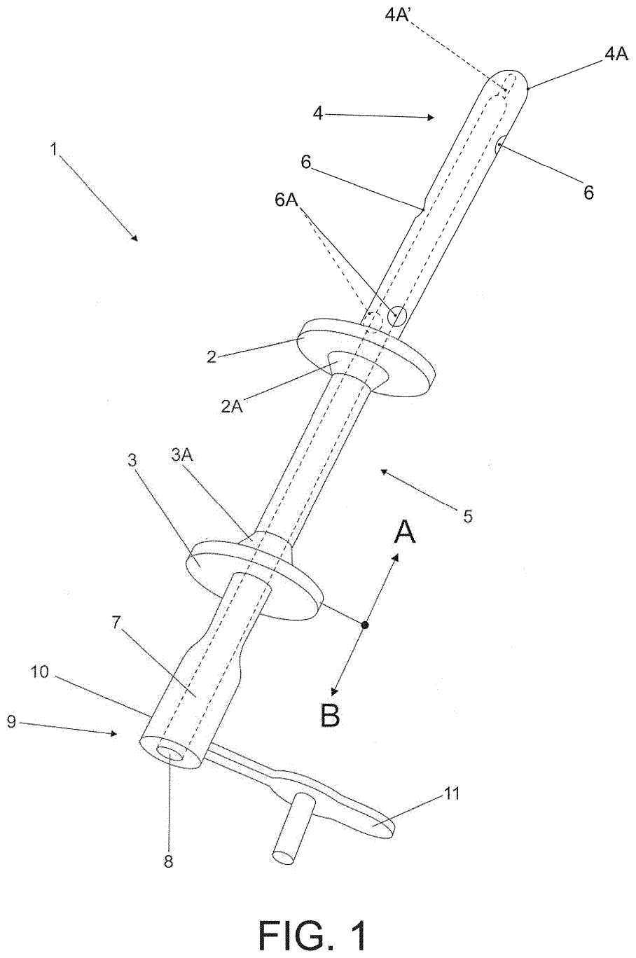

FIG. 1 shows a perspective view of the permanent, removable and self-sustainable urethral probe described herein with indication of possible adjustment movement of its distal sliding disc, such movement being indicated by the “A”-“B” arrow, where the “A” branch of the “A”-“B” arrow indicates sliding of the distal disc towards the proximal end of the probe, while the “B” branch of the same “A”-“B” arrow indicates sliding of the distal disc towards the distal end of the probe.

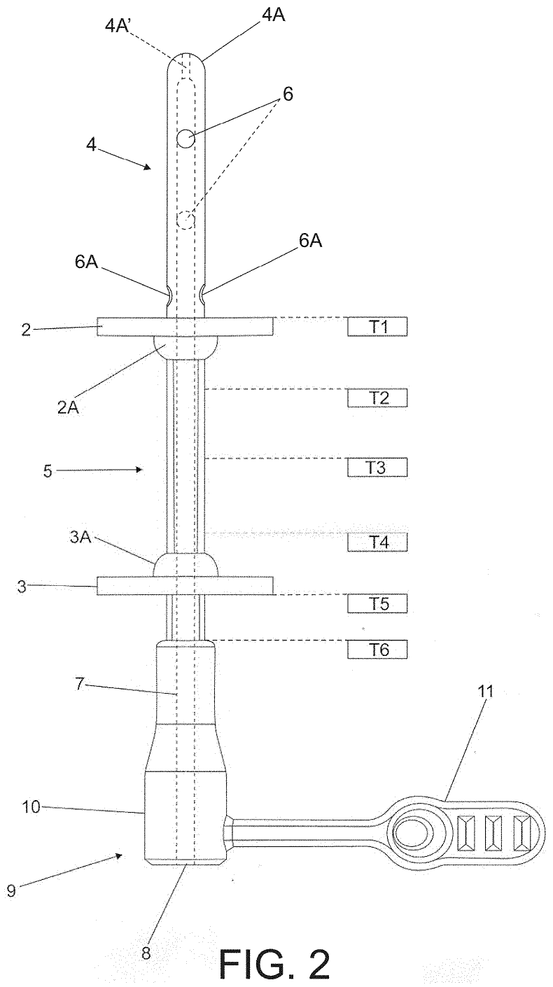

FIG. 2 shows a side view of said probe, where the progression readings/distances for the distal sliding disc are indicated.

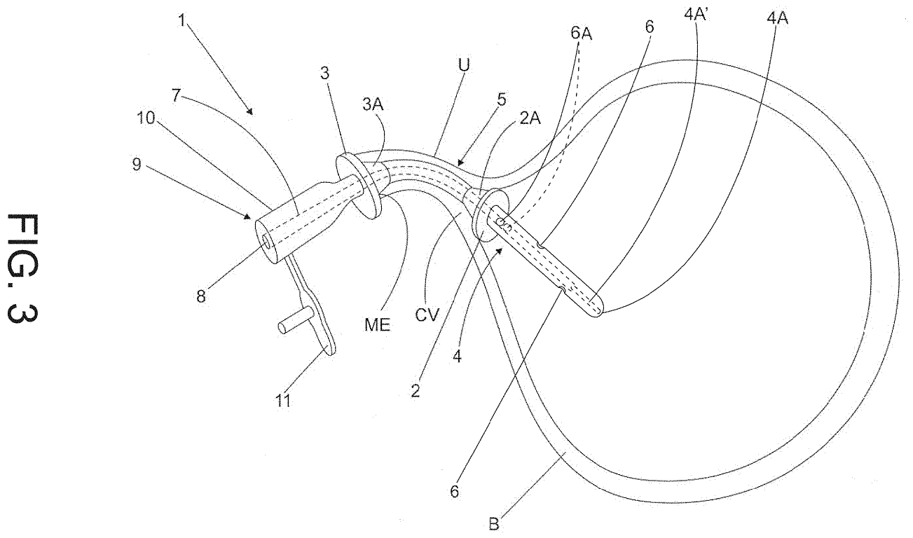

FIG. 3 shows a schematic view, which shows the practical use of the probe described herein, with said probe shown as to be inserted through the urethra of a patient and reaching the bladder.

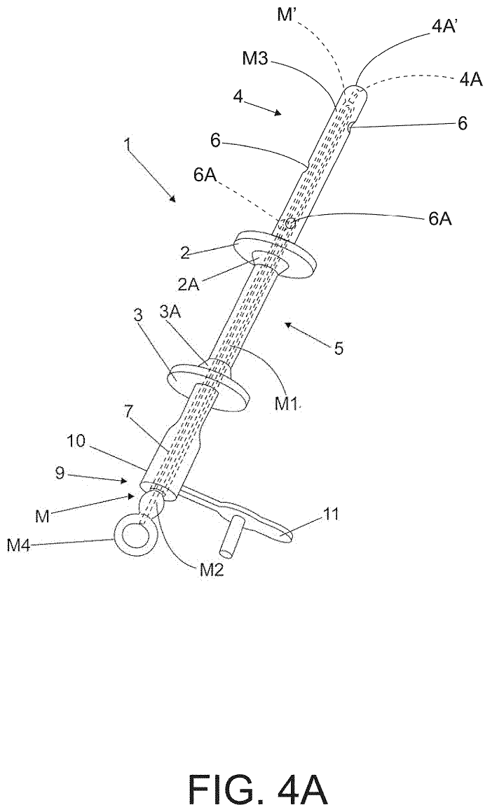

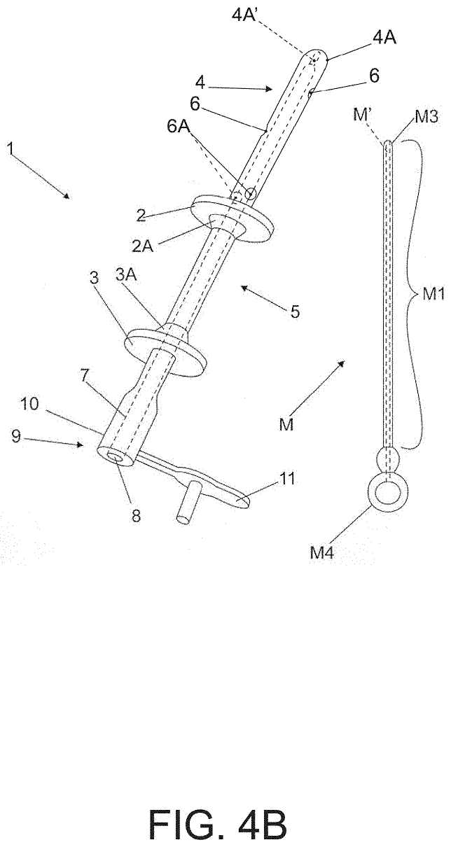

FIGS. 4A and 4B show, respectively, the probe described herein, including the chuck, and the aforementioned probe with said chuck alongside it, in order to enable introduction of the probe through the patient's urethra.

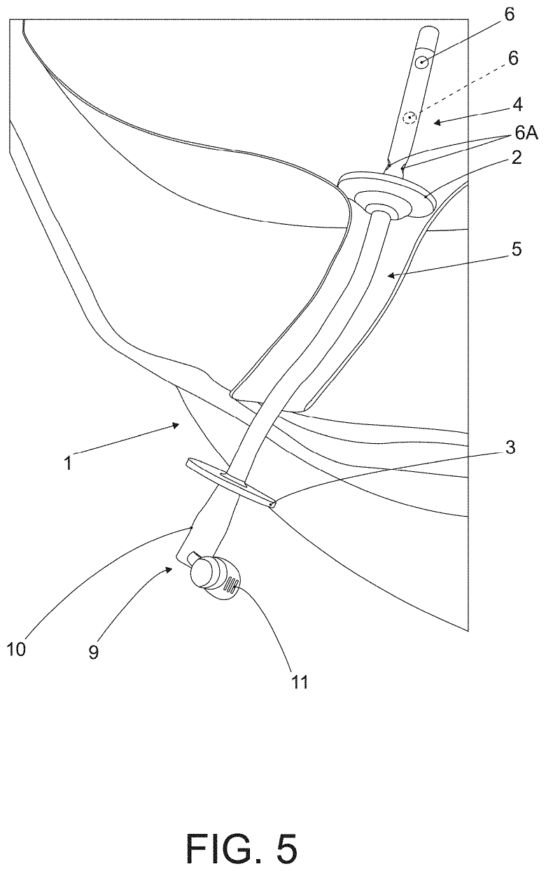

FIG. 5 shows use of the permanent, removable and self-sustained urethral probe.

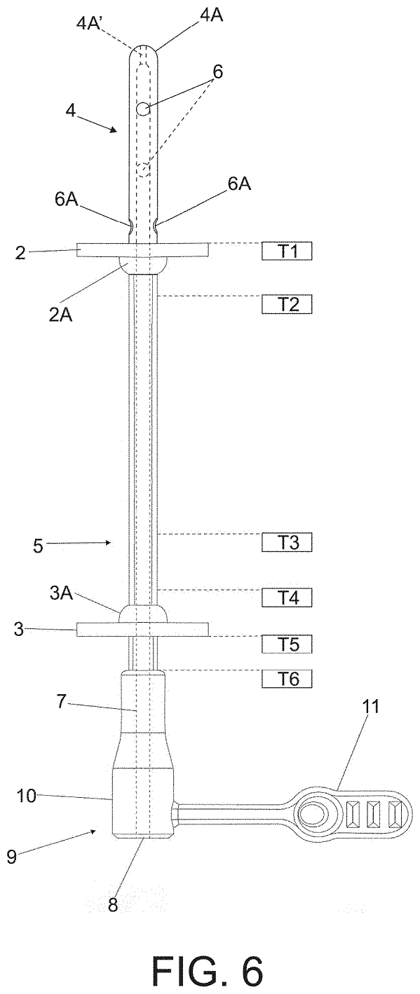

FIG. 6 shows a side view of the wheelchair developed probe where readings/distances of progression to the distal sliding disc are indicated.

The probe that is object of this invention patent application is generally indicated by reference number 1 and is manufactured in medical grade silicone including two discs, each with a central protuberance, one being a fixed proximal disc 2 which, when coupled to the bladder neck CV, provides occlusion, and also including a protuberance as indicated by reference 2A; the other being a distal disc 3, with its respective protuberance indicated by reference 3A, with said distal disc 3 being slidable and adjustable to the external female meatus or male perineal urethrostomy in order to provide full occlusion of the patient's urethra, therefore providing continence.

Protuberances 2A and 3A of discs 2 and 3 may feature a substantially conical shape, required for perfect fixation of the probe to the bladder neck CV and the external meatus of the urethra ME in the female and perineal region.

The proximal end 4 of the tubular body 5 of the probe 1 includes 4 holes 2 indicated by reference number 6, through which the urine volume contained inside the bladder B of the patient may be drained through a longitudinal channel 7, traversing the tubular body 5 from the region of the holes 6 up to the lumen 8 located on the distal end 9.

Probe 1 is defined by a tubular body 5 with four holes 6 and 6A, two of them 6A, positioned next to the proximal disc 2 and which are 2.5 cm long.

The existence of four holes is essential to avoid accumulation of crystals provenients from the urine which holds potential to obstruct the probe specially if the device extrapolates the time for replacement. Moreover, the position of the four holes can also be essential to avoid accumulation of crystals provenients from the urine.

The end portion of the proximal end 4 features a rounded tip 4A, which includes a 1 mm diameter hole (indicated as 4A′) in order to allow passage of the aforementioned guide wire (not shown), which does not integrate the claimed subject of this invention.

The distal end 9 of the probe 1 includes a connector 10 with a cover 11 and an open close mechanism that enables emptying of the bladder B when required, as may be assumed upon visualization of FIGS. 1, 2 and 3.

The probe 1 includes a chuck M (as shown on FIGS. 4A and 4B) which is to enable introduction of the probe 1 through the patient's bladder U. It comes in two sizes to allow safe insertion of the probe in its different sizes.

The chuck M features a shaft-shaped portion M1, which is dimensioned to be inserted through the tubular body 5 of the probe 1; an enlarged distal portion M2 which coincides, when touching the edge of the connector 10, with its end M3 reaching the terminal part of the proximal end 4 of the probe 1, such proximal end 4 including a 1 mm diameter hole indicated as 4A′, as previously mentioned; and a gripping portion M4, through which the chuck M is handled.

The chuck M features a channel M′ for its entire length, through which a guide wire (not shown) is eventually used in the positioning procedure of the probe 1.

It should be noted that hole 4A′, incorporated to the proximal end 4 of the probe 1 is included in full alignment with the channel M′ provided throughout the chuck M.

The state of the art to be considered useful to the understanding of this invention is to be provided by the introduction of the probe 1 through the urethra U up to the bladder neck CV, for female patients.

For male patients required to use the probe, it should be introduced via a minor perineal urethrostomy. As shown in FIG. 5.

The guide wire mentioned herein is an accessory, and is not part of this invention, since it is used eventually, in other words, in more complex situations that require easier insertion conditions for the probe.

The probe mentioned herein is indicated, in general, by reference number 1, while both discs included in the probe (discs 2 and 3) are malleable and pose no difficulties for this procedure.

One disc (proximal disc 2) remains in the internal portion of the bladder neck CV (proximal), therefore, inside the bladder B, and the other disc (distal disc 3) is placed on the external meatus of the urethra ME or in the perineal region in the male (FIG. 5).

The sliding of the disc 3 through the probe 1 allows adjustment in relation to the length of the urethra U, such adjustment being shown regarding the movement shown by arrow “A”-“B”, as shown by FIG. 1.

This mechanism works upon traction exerted by the urologist on the moment of the introduction of the probe 1. After it is fixed to the bladder neck CV (disc 2) and the external urethral meatus ME (disc 3) through the respective protuberances 2A and 3A included on discs 2 and 3 or in the male perineal region.

The probe 1 described herein may be removed or replaced at any time, functioning for six to eight months due to the material used in manufacturing and its reduced size.

Said probe is manufactured with 2 gauges (12 and 14 FR) for children, and 4 (16, 18, 20 and 22 FR) for adults.

The total length of this probe 1 is 7 cm for child models and 8 cm for adult models. A 15 cm is also available to be used in patients with special needs such wheelchairs.

In both cases, both the gauge and the size of the probe 1 may vary according to particular anatomical features of the patient.

FIG. 2 shows the probe 1 described herein, regarding indications referring to distances between discs 2 and 3, where the dashed lines T1, T2, T3, T4, T5 and T6 correspond to 0 cm, 1 cm, 1.5 cm, 2 cm, 2.5 cm and 3 cm measures, respectively.

What is claimed is:

1. A permanent, removable and self-sustainable urethral probe, comprising:
a connector having a lumen; and
a tubular body of medical grade silicone with a smooth surface, the tubular body having a proximal end with an edge that is closed by a longitudinal rounded tip, the longitudinal rounded tip traverses the tubular body from the edge of the proximal end up to the lumen located on the connector, wherein the tubular body includes two discs, one of the two discs being a fixed proximal disc and the other of the two discs being a mobile distal disc, and wherein both of the two discs include a protuberance.

2. The urethral probe according to claim 1, wherein the two discs establish fixation of the probe in relation to a bladder neck and a urethra of a patient.

3. The urethral probe according to claim 1, wherein the two discs are flexible and allow size adjustment of the probe according to a length of a urethra of a patient.

4. The urethral probe according to claim 1, wherein the fixed proximal disc is fixable to a bladder neck of the patient.

5. The urethral probe according to claim 1, wherein the mobile distal disc is fixable to an external part of a urethra of a patient on an external urethral meatus in females or perineal region in males.

6. The urethral probe according to claim 1, wherein the protuberance included on both of the two discs features a cone-shaped structure required for fixation of the probe to a bladder neck and to an external urethral meatus in females or perineal region in males.

7. The urethral probe according to claim 1, wherein the distal end further comprises an open-close mechanism that enables bladder emptying.

8. The urethral probe according to claim 1, wherein the tubular body has two holes positioned next to the proximal disc, diametrically opposite each other, the tubular body being 2.5 cm long.

9. The urethral probe according to claim 1, further comprising a disposable polypropylene chuck in two sizes, one size for child use and another size for adult use.

10. The urethral probe according to claim 1, comprising a total length of 1 to 7 cm for child use.

11. The urethral probe according to claim 1, comprising a total length of 8 cm for adult use.

12. The urethral probe according to claim 1, comprising a total length of 15 cm for patients with special needs.

13. A permanent, removable and self-sustainable urethral probe, comprising:
a tubular body having a longitudinal channel between a proximal end and a distal end;
a rounded tip closing the longitudinal channel at the proximal end;
a lumen opening the distal end of the longitudinal channel;
a first disc fixed on the tubular body at the proximal end;
a second disc movable on the tubular body along the longitudinal channel, the second disc being between the distal end and the first disc; and
two holes in the tubular body into the longitudinal channel, the two holes being positioned next to the first disc, diametrically opposite each other.

14. The urethral probe according to claim 13, further comprising:
a first protuberance on the first disc, the first protuberance facing the distal end; and
a second protuberance on the second disc, the second protuberance facing the proximal end.

15. The urethral probe according to claim 14, wherein the first and second protuberances are cone-shaped.

16. The urethral probe according to claim 13, wherein the tubular body has two additional holes positioned between the rounded tip and the two holes next to the first disc.

17. The urethral probe according to claim 16, wherein the two additional holes are diametrically opposite each other, offset from one another along the longitudinal channel, and are offset from the two holes by ninety degrees.

18. The urethral probe according to claim 13, wherein the first and second discs are configured to fix the tubular body to a patient.

19. The urethral probe according to claim 13, further comprising an open-close mechanism configured to selectively open and close the lumen.

20. The urethral probe according to claim 13, further comprising a chuck removably positioned in the longitudinal channel, the chuck having a channel through an entire length.
